(19) United States
(12) Patent Application Publication
(10) Pub. No.: US 2011/0082071 A1
(43) Pub. Date: Apr. 7, 2011

(54) DELIVERY SYSTEM FOR AN ACTIVE INGREDIENT

(76) Inventors: Amal Elabbadi, Annemasse (FR); Olivier Haefliger, Geneva (CH); Lahoussine Ouali, Vetraz-Monthoux (FR)

(21) Appl. No.: 12/988,390

(22) PCT Filed: May 14, 2009
(86) PCT No.: PCT/IB09/52011

§ 371 Date: Oct. 18, 2010

(30) Foreign Application Priority Data
May 15, 2008 (IB) PCT/IB2008/051917

(51) Int. Cl. A61K 38/02; A61K 47/04; A61K 31/715; A61K 31/37; A23L 1/226

(52) U.S. Cl. 514/1.1; 514/769; 514/54; 514/457; 426/534; 426/74; 426/72; 426/89; 426/271

(57) ABSTRACT

The invention relates to a delivery system for an active ingredient. The system provides an encapsulating material for the active ingredient, wherein the encapsulating material is formed by combining a cationic component, an anionic component and the active ingredient. The anionic component is a mixture of carbonate and phosphate moieties, wherein the molar ratio of carbonate to phosphate moieties is from 9:1 to 1:9.

### TECHNICAL FIELD

The present invention relates to a delivery system for active ingredients, a method of preparing the delivery system and the use of the delivery system to mask bitter tastes.

### BACKGROUND AND PRIOR ART

It is well known that certain consumable products in the foods, beverages and drugs industries contain bitter substances that are detrimental to the overall flavour impact of the product being consumed and which adversely affect consumer preference for such products. In order to deal with this, manufacturers have gone to great lengths to mask or even remove the offending products.

The problem is particularly acute in beverages such as beer, coffee, and soft drinks as well as many pharmaceutical products where it is believed that the presence of polyphenols, such as chlorogenic acid lactones or flavonoids, contribute significantly to bitterness perception by consumers.

Nevertheless, many polyphenols or flavonoids found in foodstuffs are beneficial anti-oxidants which, when consumed, scavenge so-called “free radicals” or modulate human or animal gene expression and so provide nutritional or health benefits to the consumer. For a more detailed understanding of the beneficial effects of flavonoids, for instance, see “Flavonoids: A review of probable mechanisms of action and potential applications”, Nijveldt et al, Am J Clin Nutr 2001; 74:418-25.

Therefore, it would be desirable to mask or otherwise inhibit the undesirable flavour perception by consumers due to such ingredients without detrimentally affecting the beneficial effect that they are known to impart.

It is also known to extract these beneficial ingredients from foodstuffs so as to provide them in an isolated form, such as a nutritional supplement, which can be consumed in order to receive the benefit directly. However, in this concentrated form, the risk of consumer rejection due to the bitterness of the product is even more acute.

Thus, it would be desirable to provide such extracts or supplements in a more palatable format with the perception of unpleasant bitterness significantly reduced or even eliminated.

JP 2003-128664 (Nagaoka Koryo KK) describes neutralizing polyphenols to the corresponding sodium, calcium, magnesium or potassium salt in order to reduce bitterness. This method forms large particles that can dramatically alter the appearance of drinks (such as tea), consequently reducing consumer preference therefor.

In JP 2003-366456 (Taiyo Kagaku KK) the bitterness and astringency in beverages and foods is said to be decreased by the addition of casein.

In JP 04-103771 (Unitika KK), a tea extract is prepared by blending tea with chitin so as to eliminate bitterness and astringency.

US-A1-2002/0188019 (Bayer Corporation) describes preparations comprising certain hydroxyflavanones which are said to mask bitter or metallic taste sensations.

U.S. Pat. No. 5,741,505 (Mars) describes using inorganic coatings to provide an oxygen barrier to increase the shelf-life of foods and pharmaceutical products. The coating does not interact with the encapsulated product.

US 2004/0180097 (Lin et al) refers to a stable and/or taste-masked pharmaceutical dosage form comprising porous apatite grains and a drug entrapped in the pores. The product is formed by contacting blank porous apatite grains, typically in the form of slurry, with a solution of the drug and evaporating the solvent of the solution in order to entrap the drug in the porous apatite grains. Thus, there will be a high concentration of the drug at or near the surface of the granules, leading to uneven distribution of the product and risk of loss of a higher proportion of the entrapped product than if the product was distributed more evenly throughout the granule.

There are also numerous products available to the public in which a liquid active ingredient, such as garlic oil, is encapsulated in a transparent shell. These capsules are very large, typically having a diameter of up to 5 to 10 mm, rendering them potentially difficult or unpleasant for some consumers to swallow whole. Such capsules would also be aesthetically unappealing for incorporation into a variety of foodstuffs or beverages.

It would thus be desirable to provide a product having a particle size diameter that is, at most, barely noticeable with the naked eye. Such particles are then suitable for incorporation into products, especially beverages, where the presence of visible particles may be undesirable.

Various studies have also shown that the beneficial effects on health due to, for instance, polyphenols can be increased by the delivery of the intact active to the digestive system, rather than within the oral cavity.

Thus, it would be desirable to protect the active ingredient in such a way that release is triggered by physical conditions present in the stomach or digestive system but which are not present during conventional storage or in the oral cavity.

Our co-pending application, WO-A1-2008/072155 discloses a carrier system in which the material to be encapsulated is both entrapped within the encapsulating material and bound thereto. This has been found to provide a stable system for delivering active ingredients, such as polyphenols, intact to the stomach or digestive system of a consumer wherein the low pH then causes the release of the active ingredient. The system is based entirely on a metal phosphate or a metal carbonate, but not a combination thereof. Nevertheless, it would be desirable to improve the loading of the active ingredient that can be achieved by such a system.

Many active ingredients are extremely sensitive to oxidation. Therefore, it would be desirable to provide an encapsulation system that is capable of protecting the active ingredients against such degradation.

It is also a known problem with various encapsulation systems that the loading of the active ingredient therein is limited and thus the provision of an encapsulation system having an improved loading is desirable.

Additionally, for certain active ingredients, it is desirable to provide a boost effect whereby, in use, the active ingredient is perceived very strongly after a certain period. It would thus be desirable to provide an encapsulation system that can provide such a boost effect.

The present invention seeks to address one or more of the abovementioned problems and/or to provide one or more of the abovementioned benefits.

### SUMMARY OF THE INVENTION

Accordingly, the present invention provides a delivery system for an active ingredient, the system comprising an encapsulating material for the active ingredient, wherein the encapsulating material comprises
(i) a cationic component,
(ii) an anionic component comprising a mixture of carbonate and phosphate moieties, wherein the molar ratio of carbonate to phosphate moieties is from 9:1 to 1:9.

The invention further provides a method of preparing an encapsulated active ingredient comprising the steps of:
(i) providing a first source of a cationic component of an encapsulating material,
(ii) providing a second source of an anionic component of the encapsulating material,
(iii) providing a third source of an active ingredient,
(iv) mixing the three sources in any order of addition,
   so as to retain the active ingredient in the encapsulating material, wherein the source of the anionic component comprises a source of carbonate ions and a source of phosphate ions in a molar ratio of carbonate to phosphate moieties of from 9:1 to 1:9.

In another aspect, the invention provides the use of an encapsulating material, as defined above, to mask, inhibit or otherwise reduce a consumer's perception of bitterness of a bitter active material.

In yet another aspect, the invention provides a consumable product comprising the delivery system defined herein.

### DETAILED DESCRIPTION OF THE INVENTION

The present invention relates to a delivery system based on an encapsulating material comprising a cationic component and an anionic component, the anionic component comprising a blend of phosphate and carbonate groups in a specific molar ratio.

The structure of the delivery system according to the present invention can be described as hybrid. That is, it can be considered to combine both a shell structure for surrounding an active material and a matrix structure throughout which the active material is distributed or dispersed. Such a system is found effectively to release the active ingredient either by mechanical defragmentation in the digestive tract and/or by pH changes in the stomach and so provides a useful mechanism for delivering active ingredients, such as nutritional or health products, intact to where they are most effective.

As will be readily understood by the person skilled in the art of encapsulation, the encapsulating product of the invention is entirely different from conventional encapsulation products, the latter typically comprising either an encapsulating shell which fully surrounds the active ingredient (the so-called “core-shell” arrangement) or a matrix throughout which the encapsulated product is distributed (such as spray-dried or extruded particulate product).

The active ingredient delivery system may be in the form of a colloidal hybrid.

It has been found that such colloidal hybrids have a large specific surface area. This is advantageous because, when the conditions are suitable for releasing the encapsulated ingredient (e.g. due to the acidic pH in the stomach), there is a greater reactive surface area available which accelerates the rupture/dissolution of the encapsulating material and so enables release of the active ingredient to occur more rapidly.

### Encapsulating Material

The encapsulating material comprises a cationic component and an anionic component wherein the anionic component.

As indicated above, the phrase “encapsulating material” denotes a material which is capable of both surrounding an active material and fixing the material within a matrix structure.

By “fixing”, it is meant that the carrier preferably forms a bond, such as a complex, with the active ingredient. Of course, other types of bond may be possible, as will be appreciated by the person skilled in the art. Nonetheless, complexing is preferred.

By “surrounding”, it is meant that the encapsulating material forms, at least partly, a protective layer or shell around the active ingredient.

It is believed that by both fixing and surrounding the active ingredient, the ingredient is homogeneously distributed throughout the carrier. This is unlike the heterogeneous distribution that is often associated with known inorganic carrier systems where the carrier is typically porous. In these systems, the active ingredient is simply entrapped within in the pores leading to a concentration of the active ingredient at or near the surface of the carrier.

The encapsulating material preferably, though not essentially, has an amorphous structure.

“Amorphous”, as defined herein, means at least partly non-crystalline (i.e., a significant part of the compound lacks a distinct crystalline structure).

Thus, it should be understood that an amorphous encapsulating material may contain amounts of microcrystalline matter that can be tolerated without meaningful effect on the gross physical characteristics of these materials or on the enhanced complexation-encapsulation benefits that they provide.

In the context of the present invention, the encapsulating material is amorphous if it contains less than about 50%, preferably less than about 40%, more preferably less than about 30%, even more preferably less than 10%, most preferably less than 5%, e.g. less than 2% by weight of crystalline material, based on the total weight of the inorganic salt.

The encapsulating material is preferably substantially water-insoluble. “Substantially water-insoluble” is defined herein as meaning a solubility of less than 10⁻³ g/cc, more preferably less than 10⁻⁴ g/cc and most preferably less than 10⁻⁵ g/cc, when measured at 20° C. in an aqueous medium having a pH of between 3 and 7.

The encapsulating material is preferably in the form of a solid at the pH typically encountered during storage.

Preferably, the encapsulating material is soluble at very low pH values. More preferably, it has a solubility of greater than 10⁻³ g/cc, more preferably greater than 10⁻² g/cc and most preferably greater than 10⁻¹ g/cc, when measured at 37° C. and pH 2 or lower.

In other words, the pH typically found inside the stomach of a consumer will cause the rupture/dissolution of the encapsulating material and the eventual release of the active ingredient encapsulated therein.

Examples of the cationic moiety suitable for use in the encapsulating material include calcium (II), magnesium (II), iron (II), iron (III), zinc (II) or mixtures thereof.

More preferably, the cationic component is either calcium (II), magnesium (II) or a mixture thereof. Most preferably, it is calcium (II).

The anionic counterion mixture or blend comprises phosphate ions and carbonate ions in a carbonate to phosphate molar ratio of 9:1 to 1:9. In the context of the present invention, the combination of the phosphate and carbonate moieties is referred to as “the anionic component”.

More preferably, the molar ratio is from 9:1 to 1:4.

Whilst a counterion based entirely on phosphate, as disclosed in our copending application WO-A1-2008/072155, has been found to generate an amorphous encapsulating material having hydrophobic splitting planes which facilitate binding of the salt to the material being encapsulated, the present invention surprisingly shows that a combination of phosphate and carbonate ions in a specific weight ratio can significantly improve the loading of the active ingredient achieved by the carrier system.

The first anionic component is a phosphate. The source of the phosphate may be any suitable salt that is soluble in water at the temperature of encapsulation. For instance, sodium hydrogen phosphate is suitable though the skilled person will readily appreciate that many other phosphate salts are also suitable.

The phosphate that is present also depends on the pH of the medium in which the encapsulation system is prepared. For instance, in strongly-basic conditions, the phosphate ion (PO₄³⁻) predominates, whereas in weakly-basic conditions, the hydrogen phosphate ion (HPO₄²⁻) is prevalent and in weakly-acid conditions, the dihydrogen phosphate ion (H₂PO₄⁻) is most common.

Thus, in the context of the present invention, the term “phosphate” is used to denote the phosphate ion, the hydrogen phosphate ion, the dihydrogen phosphate ion and mixtures thereof.

The second anionic component of the encapsulating material is a carbonate. In the context of the present invention, the term “carbonate” is used to denote the carbonate ion, CO₃²⁻, as well as the hydrogen carbonate ion, HCO₃⁻ and mixtures thereof. The source of the carbonate may be any suitable salt that is soluble in water at the temperature of encapsulation. For instance, sodium carbonate is suitable though the skilled person will readily appreciate that many other carbonate salts are also suitable.

Surprisingly, where the anionic component comprises higher amounts of the hydrogen phosphate ion or dihydrogen phosphate ion, the loading of active ingredient achievable at higher levels of carbonate is dramatically improved. Thus, in a preferred aspect, the delivery system comprises, as phosphate component, 20% or more, more preferably 30% or more, even more preferably 50% or more, most preferably 60% or more, even 70% or more HPO₄²⁻ and H₂PO₄⁻, by weight based on the total weight of phosphate.

The molar ratio of cationic component to the anionic component is preferably from 2:1 to 1:3, more preferably from 1.5:1 to 1:2.5, even more preferably from 1:1 to 1:2, most preferably from 1:1.1 to 1:1.5.

It is preferred that the carrier comprises a molar excess of the anion component over the cationic component since the resulting negatively charged carrier has a greater colloidal stability and/or is more easily dispersed in aqueous liquid media. For instance, it is envisaged that the delivery system of the invention is to be used in beverages, such as tea, coffee, cordials and the like and, for this purpose in particular, the excess negative charge is advantageous.

Additional materials may be present together with the encapsulating material to make a complex encapsulating material. Organic materials are particularly preferred. For instance, carbohydrates such as maltodextrin, cyclodextrins and chemically modified starches may also be present so as to form a complex encapsulating material.

### Active Ingredient

The active ingredient can be any compound or composition that it is desirable to fix and to encapsulate.

Nevertheless, the present invention has been shown to work surprisingly well at reducing consumer perception of undesirable flavours imparted by active ingredients whilst allowing the ingredients to be delivered intact for release in the digestive system or stomach of a consumer.

In particular, the hybrid encapsulating material has been shown to mask bitter or astringent tastes particularly effectively.

It has also been found to prevent undesirable oxidation of active ingredients, even in the case of extremely oxygen-sensitive active ingredients.

Preferred active ingredients include polyphenols, conjugated polyphenols, polyphenol polymers, coumarins, polysaccharides, lipids, organosulphur compounds, conjugated vitamins, peptides, carotenoids, proteins or mixtures thereof.

In a preferred aspect, the active material is a polyphenol.

A particularly preferred polyphenol is a glycone optionally conjugated with one or more of methyl groups, sulphates, glycosides, phosphates, acetates and/or esters.

Examples of suitable polyphenols include the family of flavonoids.

Flavonoids include (i) flavones, such as chrysin, kaempferol, rutin, quercetin, luteolin and apigenin, (ii) flavanols, such as quercetin, kaempferol, myricetin, isorhamnetin, pachypodol, rhamnazin, (iii) flavanones, such as fisetin, naringin, naringenin, hesperetin, naringenin, eriodictyol, (iv) flavan-3-ols such as (+)-Catechin, (+)-Gallocatechin, (−)-Epicatechin, (−)-Epigallocatechin, (−)-Epicatechin 3-gallate, (−)-Epigallocatechin 3-gallate, theaflavin, theaflavin-3-gallate, theaflavin 3′-gallate, theaflavin 3,3′ digallate, (v) thearubigins, (vi) isoflavones such as genistein, daidzein, glycitein, (vii) anthocyanidins such as cyanidin, delphinidin, malvidin, pelargonidin, peonidin and petunidin, (viii) polymethoxyflavones, (ix) flavans, (x) phenolic flavanoids, (xi) proanthocyanidins and (xii) isoflavonoids.

Polyphenol active ingredients are found in a variety of natural consumer products such as grapefruit juice, green tea, black tea, and coffee. See publication Bitter Taste, Phytonutrients, and the consumer: a Review, The American Journal of Clinical Nutrition, Adam Drewnowski and Carmen Gomez-Carneros December 2000, 72: 1424-1435. The encapsulating material is particularly effective when used in combination with such foodstuffs or their extracts. For instance, active ingredients comprising green tea extract or fermented tea extract are particularly suitable.

In another aspect, the active ingredient may be a colorant, more preferably a carotenoid. Examples of suitable carotenoids include beta-carotene, retinol, astaxanthin, lutein, lycopene, cryptoxanthin and zeaxanthin. Colorants such as these are typically used in translucent beverages. Upon storage, the colorant can sometimes settle to give an undesirable difference in colour shades throughout the beverage. Furthermore, vigorous shaking is then needed to redisperse the colorant evenly throughout the beverage. Using the hybrid encapsulating system to fix and envelop the colorant, the very small encapsulated particles have been found to remain evenly suspended throughout the beverage, even upon storage.

Other suitable active ingredients include phenolic acids, tocopherol phosphates, tocopherol acetates, stilbenes, resveratrols, curcumins, vitamins, 6-gingerols, furanocoumarins, bergamottins, triterpens (limonoids), tannins, punicalagins, punicocides, ellagic acids, lignans, procyanidins, pycnogenols, phytosterols, glucosynolates, hydrolyzed glucosinolates, isothiocyanates, sulphoraphanes, glutathiones, ergothioneines, lipoic acids, sphingolipids and butyrates.

Yet further suitable active ingredients include oils rich in polyunsaturated fatty acids. Such oils typically comprise at least 5 wt. %, preferably at least 10 wt. %, more preferably at least 25 wt. %, and most preferably at least 35 wt. % polyunsaturated fatty acids based on the total weight of the oil.

The oil rich in polyunsaturated fatty acids is preferably an oil rich in omega-3 fatty acids.

More preferably, the polyunsaturated fatty acids are selected from eicosapentaenoic acid (EPA), docosahexaenoic acid (DHA), arachidonic acid (ARA), α-linolenic acid, linoleic acid, and a mixture of at least two thereof. DHA and EPA are most preferred.

It is preferred that the oil is mixed with a vegetable oil derivative, such as a triglyceride oil. A particularly preferred range of commercially available oils are sold under the tradename Neobee® (ex Stepan).

A preferred weight ratio of oil rich in polyunsaturated fatty acids to vegetable oil derivative is from 70:30 to 99:1, more preferably from 80:20 to 95:5.

In a highly preferred embodiment, the oil mixture is then emulsified using any suitable emulsifying agent. Preferably the emulsifier is food grade, more preferably it is a sugar ester. The emulsified oil is found to mix more readily with the carrier system and so provides a more stable product.

The active ingredient may comprise a flavouring or perfuming ingredient, compound or composition.

The flavouring or perfuming material defines a variety of flavour and fragrance materials of both natural and synthetic origin. They include single compounds and mixtures. Natural extracts can also be encapsulated; these include e.g. citrus extracts, such as lemon, orange, lime, grapefruit or mandarin oils, or essential oils of spices, amongst other. Particularly preferred active materials in this class for encapsulation are flavour compositions containing labile and reactive ingredients such as berry and dairy flavours.

Further specific examples of such flavour and perfume components may be found in the current literature, e.g. in Perfume and Flavour Chemicals, 1969, by S. Arctander, Montclair N.J. (USA); Fenaroli's Handbook of Flavour Ingredients, CRC Press or Synthetic Food Adjuncts by M. B. Jacobs, van Nostrand Co., Inc. They are well-known to the person skilled in the art of perfuming, flavouring and/or aromatizing consumer products, i.e. of imparting an odour or taste to a consumer product.

Where desired, the flavour or perfume ingredient, compound or composition can be emulsified in order to improve its incorporation into the delivery system. Many suitable emulsifiers exist for this purpose, such as for instance citrem and gum Arabic, and they are well known to the person skilled in the art of perfuming and flavouring.

Due to its hybrid nature and the careful ratio balance of cationic and anionic components, the delivery system of the present invention allows for very heavy loading of the active ingredient onto and into the encapsulating material.

Thus, the active component may comprise up to 80% by weight of the total weight of the delivery system.

The advantage is that less encapsulating material is need in order to deliver the required amount of active ingredient thereby increasing cost effectiveness when compared to traditional encapsulation systems.

The delivery system may be in solid, semi-solid or liquid form.

If solid, it is preferably in the form of particles.

The particle size may vary but is preferably from 0.05 to 1000 μm, more preferably from 0.1 to 500 μm, most preferably from 0.1 to 100 μm.

In the context of the present invention, “particle size” is defined as the arithmetic mean diameter determined by conventional light scattering experiments.

The particle size is particularly important insofar as it determines whether the particle can be used in food or beverage products where visibility of such particles is undesirable. It has been found that the delivery system according to the invention enables the preparation of much smaller particles than typically possible with conventional encapsulated products. This renders the particles more suitable for applications where visibility of the particles is desired to be minimised.

Preferably at least 90%, more preferably at least 95% and most preferably 97%, e.g. 99% by number of the particles have a particle size within the range of from 0.05 to 1000 μm, more preferably from 0.1 to 500 μm and most preferably from 0.1 to 100 μm.

It has been discovered that the particles in the delivery system of the present invention typically have a much more homogeneous size than those in traditional encapsulation systems. Homogeneously sized particles are desirable from an aesthetic viewpoint and, moreover, allow for a more regular dosage of the active ingredient.

If the delivery system is in liquid form, it is preferably provided as an aqueous dispersion.

The active ingredient delivery system may be further encapsulated. A further encapsulation of the active ingredient delivery system can be highly desirable since it enhances the oxidative stability of the delivery system upon storage.

A first preferred encapsulating system is a glassy matrix within which the active ingredient delivery system is held. More preferably the encapsulation system is a glassy carbohydrate matrix. The carbohydrate matrix ingredient preferably comprises a sugar derivative, more preferably maltodextrin.

Particularly preferred maltodextrins are those with a DE of from 10 to 30, more preferably from 15 to 25, most preferably from 17 to 19.

Typically, the active ingredient delivery system is admixed with a carbohydrate matrix material and an appropriate amount of a plasticizer, such as water, the mixture is heated within a screw extruder to a temperature above the glass transition temperature of the matrix material so as to form a molten mass capable of being extruded through a die and then the molten mass is extruded using established processes, such as described in the prior art. See, for instance, patent application WO 00/25606 or WO 01/17372 and the documents cited therein, the contents of which are hereby included by reference.

If desired, further carbohydrate matrix components may be present to improve yet further the antioxidant barrier properties.

Other suitable encapsulation systems are described in, for examples, U.S. Pat. No. 4,610,890 or U.S. Pat. No. 4,707,367, the contents of which are included by reference.

### Preparation

An encapsulating material according to the invention can be prepared in any suitable manner known to the person skilled in the art. Typically, it is prepared in situ at the same time as binding and encapsulating the active ingredient. For instance, the delivery system can be prepared by precipitation of the cationic and anionic components in the presence of a liquid solution containing the active ingredient (e.g. a polyphenol).

In one preferred aspect, the delivery system can be prepared by co-precipitation of inorganic salts and active ingredient. This is particularly advantageous for active ingredients that are poorly water insoluble. The precipitation is typically carried out by introducing separate sources of the (i) metal cation, (ii) the anionic counterion blend and (iii) the active material to be encapsulated into a mixing zone and causing a precipitation-encapsulation process to occur.

Nevertheless, it has been found possible to combine sources (i) and (iii). For instance, where the active ingredient is present in the form of an aqueous solution such as a water/organic solvent solution, a dispersion or an oil-in-water emulsion, sources (i) and (iii) can be combined using emulsifiers, preferably food-grade emulsifiers.

Surprisingly, the loading of the encapsulating material of the present invention can be improved when the preparation occurs in the presence of an acid. It is particularly desirable that the pH during processing does not exceed 10, more preferably does not exceed 9, and most preferably does not exceed 7. Any acid suitable for this purpose can be used. For instance, 1M HCl is found to be effective, though the skilled person will be aware of the very large number of acids that are also suitable.

The acid may be added at any point during the preparation process. For instance, excellent results are achieved when it is provided with the source of the anionic component of the encapsulating material. In this case it is desirable that the acid reduces the pH of the source by about 0.5, more preferably by 1, even more preferably by 1.5. Surprisingly, this has been found to cause a very significant increase in the loading of the active ingredient in the delivery system.

It is also advantageous to reduce the pH since certain active ingredients, of which green tea extract is a notable example, can degrade at higher pH values.

Once formed, if it is desired to obtain a solid product, it can be dried in any suitable manner. The skilled person will be aware of the numerous ways of drying that are suitable for use in the present invention though it is preferred to avoid harsh methods of drying in order to reduce the risk of disrupting the structure of the delivery system and consequent leakage of the active ingredient therefrom.

### EXAMPLES

The invention will now be described with reference to the following examples. It is to be understood that the examples are illustrative of the invention and that the scope of the invention is not limited thereto.

All amounts are % by weight unless otherwise indicated.

### Example 1

### Preparation of a Delivery System According to the Invention

All the solutions were prepared in Millipore water unless otherwise stated. Aqueous calcium chloride (0.1M) and a 2 wt % aqueous solution of green tea extract (ex Naturex), herein referred to as “GTE”, were simultaneously introduced into a first mixing chamber and stirred until homogenous. Each material was introduced at a flow rate of 2.5 ml/minute. The mixture was then transferred into a second mixing chamber, into which a combined mixture of aqueous sodium hydrogen phosphate (0.08M) and aqueous sodium carbonate (0.02M) was introduced at 2.5 ml/minute. The resulting solution was mixed yielding a final product with equimolar amounts of the cationic component (Ca²⁺) and anionic component (PO4³⁻ and CO3²⁻). The pH of the solution was 6.6.

The mixture was filtered under vacuum, washed 3-fold with 2 ml of water and allowed to dry at ambient temperature yielding a powdered product.

The product was analysed and found to comprise green tea extract bound to and dispersed throughout the calcium-phosphate-carbonate matrix.

To calculate the loading of GTE encapsulated in the delivery system, 0.098 g of the powdered product was firstly dissolved in 1.5 ml of 0.3M HCl solution under sonication. The suspension was then centrifuged and the supernatant removed for HPLC analysis. The remaining solid was dissolved in 1 ml of 0.3M HCl under sonication, then centrifuged, and the supernatant removed for HPLC analysis.

Using standard HPLC measurement analysis, the loading of green tea extract was calculated to be 5.4 wt %, based on the total weight of the delivery system.

### Example 2

### Influence of an Excess of the Cationic Component

Example 1 was repeated except that the flow rate of calcium chloride was increased to 3.5 ml/minute, giving a ratio of cationic component to anionic components in the encapsulating material of 1.4:1.

The resulting powdered product was prepared and analysed by HPLC in the manner described in example 1. The loading of green tea extract was found to decrease significantly compared to example 1.

### Example 3

### Influence of an Excess of the Anionic Component

Example 1 was repeated except that the flow rate of the sodium hydrogen phosphate/sodium carbonate mixture was increased to 3.5 ml/minute, giving a ratio of cationic component to anionic component of 0.7:1.

The resulting powdered product was prepared and analysed by HPLC in the manner described in example 1. The loading of green tea extract was found to increase by more than 35% compared to the loading of example 1.

The examples demonstrate that the significant benefit of an excess of the anionic component over the cationic component.

### Example 4

### Influence of the Molar Ratio of Carbonate to Phosphate Ions

Delivery systems according to example 1 were prepared with carbonate to phosphate molar ratios as follows:

TABLE 1

| | Carbonate:Phosphate |
|---|---|
| Sample | (molar ratio) |
| 1 | 10:90 |
| 2 | 20:80 |
| 3 | 30:70 |
| 4 | 40:60 |
| 5 | 50:50 |
| 6 | 60:40 |
| 7 | 70:30 |
| 8 | 80:20 |
| 9 | 90:10 |

The resulting powdered products were prepared for HPLC analysis and the loading calculated in the manner described in example 1. The results are given in the following table:

TABLE 2

| | GTE loading in |
|---|---|
| Sample | the powder (mg/g) |
| 1 | 13.0 |
| 2 | 48.9 |
| 3 | 86.3 |
| 4 | 61.7 |
| 5 | 38.6 |
| 6 | 21.4 |
| 7 | 11.1 |
| 8 | 9.0 |
| 9 | 3.6 |

The results demonstrate that excellent loading is achieved when the molar ratio of carbonate to phosphate ions is from about 20:80 to about 50:50.

### Example 5

### Influence of the pH

The effect of modifying the pH during processing was evaluated as follows:

Sample 8 from example 4 was prepared except that the pH of the starting phosphate-carbonate mixture was reduced from 10.7 to 9.4 using 1M HCL prior to addition to the second mixing chamber.

The resulting powdered product was prepared for HPLC analysis in the manner described in example 1 above. Standard HPLC analysis gave the following results:

TABLE 3

| | pH of source of anionic | GTE loading in the |
|---|---|---|
| Sample | component | powder (mg/g) |
| 8 | 10.7 | 9.0 |
| 8’ | 9.4 | 78.6 |

The results demonstrate that the loading of GTE can be significantly increased when the process of encapsulation is performed under more acidic conditions.

Moreover, excellent loading of active ingredient is shown to be achieved in delivery systems comprising a large excess of the carbonate moiety compared to the phosphate moiety.

### Example 6

### Encapsulation of a Flavour Oil

Cinnamaldehyde oil was encapsulated as follows:

A first stock solution comprising 4.85 g cinnamaldehyde and 0.15 g citrem was prepared with stirring. A second stock solution comprising 8.48 g Na₂CO₃, 2.72 g Na₃PO₄ and 1.42 g Na₂HPO₄ in 500 ml Millipore water was then prepared with stirring.

1.05 g of the first stock solution was mixed with 49 g of the second stock solution under high shear for 1 minute (Ultra Turrax, 24000 rpm). The resulting emulsion was then added to 50 ml of 0.3M CaCl₂ under mechanical stirring for 1 hour and stored at ambient temperature for 24 hours to allow a phase separation of the capsules and the water. Finally, the water was removed and the capsules dried at ambient temperature. The dry capsules are referred to as sample A.

A third stock solution comprising 2.93 g of the first stock solution mixed with 0.15 g ethylcellulose was prepared.

2 g of the third stock solution was mixed with 48 g of the second stock solution under high shear for 30 seconds (Ultra Turrax, 24000 rpm). The resulting emulsion was then added to 50 ml of 0.3M CaCl₂ under mechanical stirring for 1 hour and stored at ambient temperature for 24 hours to allow a phase separation of the capsules and the water. Finally, the water was removed and the capsules dried at ambient temperature. The dry capsules are referred to as sample B.

To ascertain the loading of cinnamaldehyde in samples A and B, the capsules were first ground in a mortar. 50 mg of capsules of each sample were extracted with 5 ml of an extraction solvent, consisting of ethyl acetate containing 150.4 μg/ml of dibromobenzene as internal standard, in a 10 ml glass bottle. Extraction was performed firstly under ultrasonication for 15 min and then under magnetic stirring for 20 minutes.

GC-FID method: AZE-DAM.

The calibration solution containing 151.5 μg/ml cinnamaldehyde gave the following results:
Retention time of internal standard: 10.822 min
Retention time of cinnamaldehyde: 12.852 min
Coefficient K for the couple (dibromobenzene/cinnamaldehyde)=2.316

The calculated loading of cinnamaldehyde was 2.1 wt % and 9.5 wt % for samples A and B respectively.

Thus the delivery system was shown to encapsulate cinnamaldehyde effectively.

1.-18. (canceled)

19. A delivery system for an active ingredient, the system comprising an encapsulating material for the active ingredient, wherein the encapsulating material comprises
(i) a cationic component, and
(ii) an anionic component comprising a mixture of carbonate and phosphate moieties present in a molar ratio of carbonate to phosphate moieties of from 9:1 to 1:9.

20. The delivery system as claimed in claim 19, wherein the cationic component is calcium, magnesium, iron (II), zinc, selenium, copper, aluminium, or mixtures thereof.

21. The delivery system as claimed in claim 19, wherein the cationic to anionic components are present in a molar ratio of from 2:1 to 1:3.

22. The delivery system as claimed in claim 21, wherein the encapsulating material is amorphous.

23. The delivery system as claimed in claim 19, wherein the molar ratio of carbonate to phosphate moieties is from 9:1 to 1:4.

24. The delivery system as claimed in claim 19, wherein the active ingredient is a polyphenol, conjugated polyphenol, polyphenol polymer, coumarin, polysaccharide, lipid, organosulfur compound, conjugated vitamin, peptide, carotenoid or protein.

25. The delivery system as claimed in claim 19, wherein the active ingredient is an emulsified oil rich in polyunsaturated fatty acids.

26. The delivery system according to claim 19, wherein the system is further encapsulated.

27. A method of preparing an encapsulated active ingredient comprising the steps of:
providing a first source of a cationic component of an encapsulating material,
providing a second source of an anionic component of the encapsulating material, wherein the source of the anionic component comprises a source of carbonate ions and a source of phosphate ions in a molar ratio of carbonate to phosphate moieties of from 9:1 to 1:9,
providing a third source of an active ingredient,
mixing the three sources in any order of addition to form the encapsulating material with the active ingredient retained therein.

28. The method according to claim 27, wherein the cationic component is mixed with the active ingredient prior to mixing with the anionic component.

29. The method according to claim 27, which further comprises adding an acid during the preparation.

30. The method according to claim 29, wherein the acid is provided together with the source of the anionic component of the encapsulating material.

31. A nutritional, nutraceutical or pharmaceutical product comprising the delivery system as claimed in claim 19.

32. A food or beverage product comprising the delivery system as claimed in claim 19.

33. A method of using an amorphous metal salt to mask, inhibit or reduce bitterness perceived by a consumer of an active ingredient in a delivery system as defined in claim 24.

34. A method of masking, inhibiting or reducing bitterness perceived by a consumer of an active ingredient of a polyphenol, conjugated polyphenol, polyphenol polymer, coumarin, polysaccharide, lipid, organosulfur compound, conjugated vitamin, peptide, carotenoid or protein, which comprises incorporating the active ingredient in a delivery system as defined in claim 19.
